(19) United States
(12) Patent Application Publication
(10) Pub. No.: US 2024/0335178 A1
(43) Pub. Date: Oct. 10, 2024

(54) METHOD AND APPARATUS FOR AUTOENCODER-BASED ANOMALY DETECTION IN MEDICAL IMAGING SYSTEMS

(71) Applicant: CANON MEDICAL SYSTEMS CORPORATION, Tochigi (JP)

(72) Inventors: Jeffrey KOLTHAMMER, Vernon Hills, IL (US); Kent BURR, Vernon Hills, IL (US); Yi QIANG, Vernon Hills, IL (US)

(73) Assignee: CANON MEDICAL SYSTEMS CORPORATION, Tochigi (JP)

(21) Appl. No.: 18/297,112

(22) Filed: Apr. 7, 2023

(51) Int. Cl. A61B 6/00; G16H 40/40

(52) U.S. Cl. CPC A61B 6/586; A61B 6/582; G16H 40/40; A61B 6/037

(57) ABSTRACT

A method for detecting an anomaly related to a medical imaging device includes acquiring data from a plurality of detectors of the medical imaging device, applying the acquired data to a first autoencoder, and detecting, based on outputs from the first autoencoder, an anomaly related to the medical imaging device.

### FIELD

This disclosure relates to detecting anomalies in a medical imaging system. The detection is based on an autoencoder that is trained to identify defects, malfunctions, and/or changes occurring in the medical imaging system.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Medical imaging systems, such as Positron Emission Tomography (PET) scanners, are widely used for diagnosis and clinical interventions. Before subjecting a patient to the hazard of medical radiation exposure with such a system, it is particularly important to ensure there are no defects, and to establish a known-good operating state. Scintillator-based, multichannel, gamma-ray detectors are typically used in a PET scanner. Due to the complexity in the structure and manufacturing process of such detectors, it is not uncommon for defects to occur that can impair the performance of the whole system. Only some of those defects are common and known, the rest may be novel and surprising.

Thus, it is conventional to check the detector performance daily by using the detectors to collect data from certain well-known radiation sources, and then produce a map of those detectors for visual inspection. More specifically, detector characteristics such as maps of counts per detector element, energy per detector, uniformity per detector region, etc. are constructed so as to be interpreted by a human or by an empirically designed algorithm. All of the known approaches require a priori knowledge of the meaning of the detector maps.

Furthermore, calibrations and corrections are often implemented through software computation based on data from a variety of sources and procedures to achieve uniform and stable performance. It is thus desirable to test the efficiency of those calibrations and corrections, and to spot an underperforming calibrating/correcting measure.

Therefore, methods and apparatus are desired to automatically learn and detect anomalies in a medical imaging system, including defects in various hardware and software modules, whether or not there is a priori knowledge of the defects.

### SUMMARY

The present disclosure relates to a method for detecting an anomaly related to a medical imaging device. The method comprises acquiring data from a plurality of detectors of the medical imaging device, applying the acquired data to a first autoencoder, and detecting, based on outputs from the first autoencoder, an anomaly related to the medical imaging device.

The disclosure additionally relates to an apparatus for detecting an anomaly related to a medical imaging device. The apparatus comprises processing circuitry. The processing circuitry is configured to acquire data from a plurality of detectors of the medical imaging device, apply the acquired data to an autoencoder, and detect, based on outputs from the autoencoder, an anomaly related to the medical imaging device.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIG. 1 shows a schematic of an autoencoder which can be used to implement various embodiments of the present disclosure;

FIG. 2 shows a non-limiting exemplary process flow overview for a method of detecting an anomaly based on an autoencoder, according to one embodiment of the present disclosure;

FIG. 3 shows a non-limiting example of a flow chart for an anomaly detection method in which an autoencoder is trained and operated with respect to data generated under different operating conditions, according to one embodiment of the present disclosure;

FIG. 4 shows a non-limiting example of a flow chart for an anomaly detection method in which an autoencoder is trained and operated with respect to data obtained with different processing ways, according to one embodiment of the present disclosure;

FIG. 5 shows a non-limiting example of a flow chart for an anomaly detection method in which an autoencoder is trained and operated with respect to data produced at different time points, according to one embodiment of the present disclosure;

FIG. 6 shows a non-limiting example of a flow chart for a method of automatically generating and applying high confidence normal training datasets in training an autoencoder with iteration, according to one embodiment of the present disclosure;

FIG. 7A shows a non-limiting example of a specific change which occurs in a PET system, according to one embodiment of the present disclosure;

FIG. 7B shows a non-limiting example of a flow chart for a method of identifying the specific change illustrated in FIG. 7A and correcting its impacts, according to one embodiment of the present disclosure;

FIG. 8A is an illustration of a perspective view of PET scanner apparatus according to one embodiment of the present disclosure; and

FIG. 8B is a schematic of PET scanner apparatus and associated hardware, according to one embodiment of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting.

For example, the order of discussion of the different steps as described herein has been presented for clarity sake. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an” and the like generally carry a meaning of “one or more,” unless stated otherwise.

Aspects of this disclosure are directed to a method and apparatus for identifying anomalies based on unsupervised machine learning, which is achieved through an autoencoder, for example. The autoencoder is a particular type of artificial neural network that is configured to identify the salient characteristics of its input data. For example, used in facial recognition, it can identify the key measurements of a face image without being told beforehand what those measurements are. Used in medical imaging, it can identify key measurements of a lung image to estimate whether the image is after inhalation or exhalation, for example. One application of autoencoders is to analyze many datasets that should be similar, and then identify anomalies when one is not. This approach has been used in place of manual defect inspection in manufacturing applications, for example, using video of textile production, images of wind turbine performance data, and photographs of manufactured glass, or printed circuits.

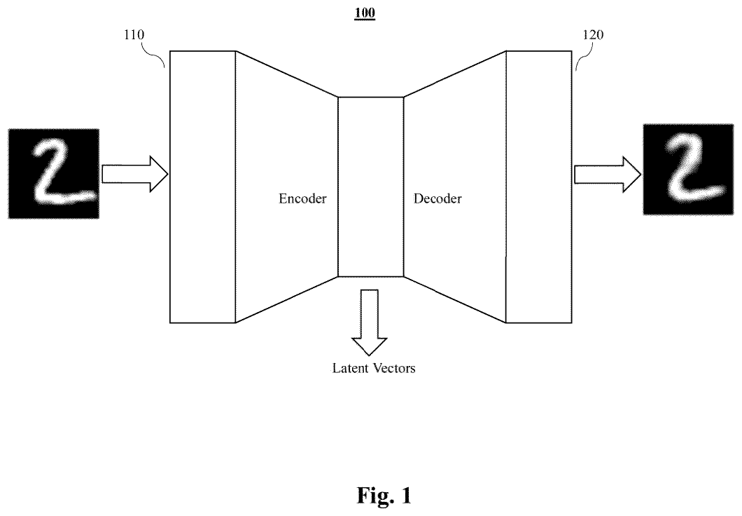

FIG. 1 shows a schematic of an autoencoder that can be used to implement various embodiments of the present disclosure. The autoencoder 100 includes an encoder 110 as an input stage and a decoder 120 as an output stage. The encoder 110 compresses its inputs into a representation in a lower-dimension of latent space (latent vectors). The decoder 120 reconstructs data from the latent space back to the original image of the original dimension. By attempting to match its outputs to its inputs, the autoencoder 100 learns to grasp relevant information and ignore insignificant data.

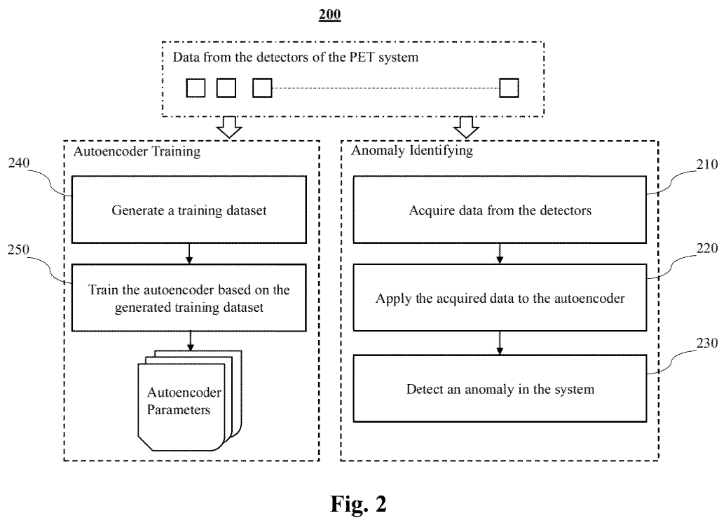

FIG. 2 shows a non-limiting exemplary process flow overview for a method of detecting an anomaly based on an autoencoder, according to one embodiment of the present disclosure.

In step 210, data (for example, PET detector maps) is acquired from detectors of a PET system during a well-known process, for example, a flood histogram of a point source or a lutetium background spectrum.

In step 220, an autoencoder is applied to the acquired data.

In step 230, outliers are identified based on the outputs of the autoencoder. The identified outliers might indicate “misbehaving” detectors.

During an autoencoder training phase, the autoencoder learns to identify the outliers. In step 240, a training dataset is generated from the data acquired from the detectors.

In step 250, the autoencoder learns based on the generated training dataset. When the training is completed, the learned autoencoder parameters are stored for use in the anomaly identification process.

The process of FIG. 2 is appliable not only to detecting defects in the detectors, but also to detecting defects in other modules of the PET system. Moreover, the process can be used to detect defects in a variety of components of medical imaging systems based on scintillator-based detectors, solid-state detectors, or flat-plane detectors, including but not limited to, Single Photon Emission Computerized Tomography (SPECT) systems and X ray imaging systems (such as Computed Tomography scanners (CTs)).

In a non-limiting example, the autoencoder can learn a flood map in an X-ray imaging device and locate dead pixels. As another example, an energy map in a SPECT system can be learned by the autoencoder to identify the failure of various photosensors. In another non-limiting example, an autoencoder can be trained on a flood image from a 2D SPECT panel with a collimator attached, so as to check both the efficiency of the panel and the alignment of the collimator. By training and running an autoencoder on a CT sinogram obtained from a quality assurance phantom scan, the performance of a plurality of parts of the CT scanner can be checked, including the detectors, the rotation alignment, and the projection/sinogramming computation hardware.

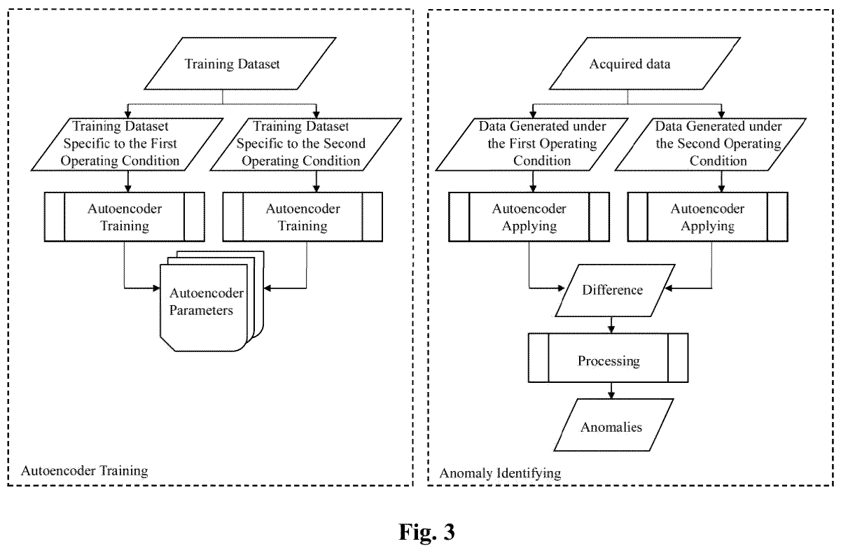

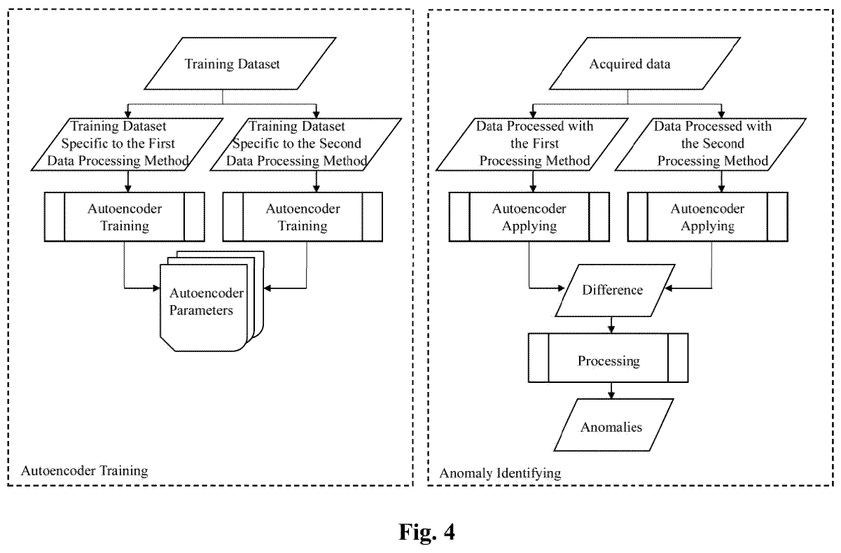

Instead of “raw” data, the method 200 can be applied to “processed” data derived from various computation processes. Using an autoencoder trained with data generated by a software procedure, the performance quality of that procedure can be characterized. In a non-limiting example, an autoencoder can learn and operate with respect to a map of calibrated data, so that both hardware performance and calibration functionality can be examined. In another non-limiting example, after being trained with a map of calibrated data from a multichannel CZT detector, an autoencoder can check whether calibration parameters are appropriately selected, whether the calibration is operating as designed, etc. In the following, two preferable embodiments for examining the efficiency of a calibration will be described with reference to FIGS. 3 and 4.

FIG. 3 shows a non-limiting example of a flow chart for a method of detecting an anomaly in which an autoencoder is trained and operated with respect to data generated under different operating conditions, according to one embodiment of the present disclosure.

Under different operating conditions, detectors can present significant individual variance in their characteristics. Normally, many kinds of calibration measures are taken to ensure a uniform output despite the varying operating conditions. For example, calibration is conducted to make the detectors work well under both high-count rate and low-count rate conditions. In the embodiments shown in FIG. 3, the acquired detector maps are split into detector maps generated under a first operating condition (e.g., a high-count rate) and detector maps generated under a second operating condition (e.g., a low-count rate). The autoencoder is applied to the two types of detector maps, respectively. A difference between the outcomes of the autoencoder with respect to the two different operating conditions can indicate errors in the calibration parameters. Preferably, although not necessary, the autoencoder can be trained independently with training datasets specific to the high-count rate and the low-count rate conditions, so as to avoid interference.

FIG. 4 shows a non-limiting example of a flow chart for a method of detecting an anomaly in which an autoencoder is trained and operated with respect to data obtained with different processing methods, according to one embodiment of the present disclosure. Similar to the example described with reference to FIG. 3, the acquired data can be split into detector maps generated from data processed by a first method (e.g., using a high-energy window) and detector maps generated from data processed by a second method (e.g., using a low-energy window) are acquired respectively. The autoencoder is applied to the two types of detector maps, respectively. A difference between the outputs of the autoencoder with respect to the two different processing methods can indicate errors in the calibration parameters. Again, in order to avoid interference, the autoencoder can be trained independently with training datasets specific to the high-energy window and the low-energy window.

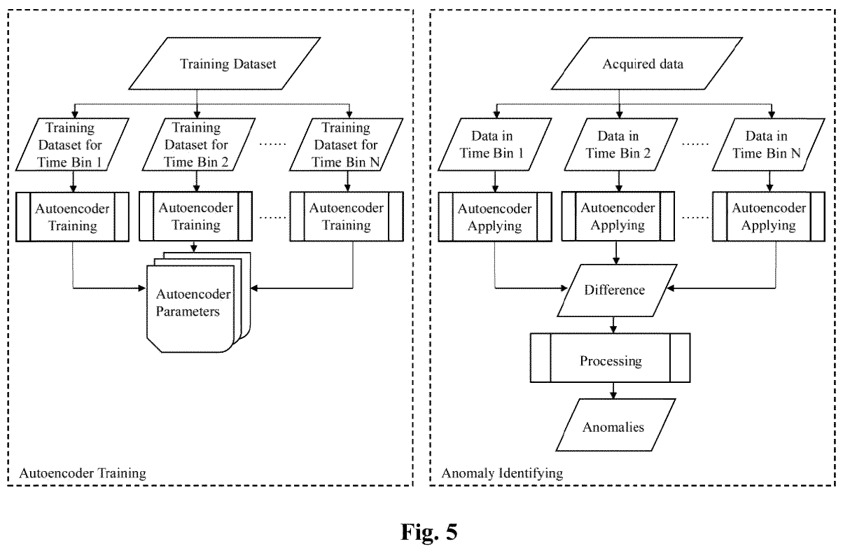

FIG. 5 shows a non-limiting example of a flow chart for a method of detecting an anomaly in which an autoencoder is trained and operated with respect to data produced at different time points, according to one embodiment of the present disclosure. As shown in FIG. 5, the obtained detector maps are accumulated in a time sequence. This can be done, for example, by real-time binning of list-mode data in a PET system, frames in a SPECT system, or fluoro-mode data in an X-ray imaging system. The data can be collected, for example, during routine quality assurance scans (using point sources, line sources, phantoms, background radiation, or a combination thereof), or during clinical scans of patients.

Then, the autoencoder can be applied to data divided into each time bin. In one embodiment, the outputs for a time bin can be compared against a reference standard to identify abnormal behavior (e.g., “drop-outs”) that occurs in time. The reference standard can be developed as a system calibration parameter, from a phantom scan or from background radiation, or as an average of many pervious patient scans, for example.

Alternatively, instead of using a reference standard, the outputs for many time bins can be compared to each other. In one example, the outputs from the autoencoder for each time bin are compared directly to identify abnormal behaviors. In another example, various processing such as clustering are applied to the outputs or the difference between the outputs for two time bins.

In one embodiment, the anomaly detection process shown in FIG. 5 is implemented at regular intervals. For example, the process is repeated every several seconds (e.g., 1 second, 5 seconds, etc.) during a quality assurance scan or a clinical scan. Thus, the system is monitored for anomalies in a real-time manner.

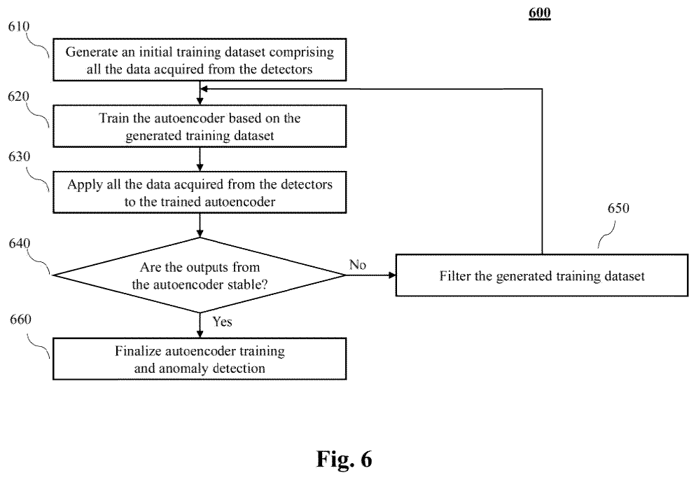

FIG. 6 shows a non-limiting example of a flow chart for a method of automatically generating and applying high confidence normal training datasets in training an autoencoder using an iterative process according to one embodiment of the present disclosure. Unlike FIGS. 2-5 in which training and operating of the autoencoder are shown in separate procedures, FIG. 6 illustrates a preferable embodiment in which an autoencoder operates iteratively on a set of input data to automatically learn and identify outliers.

The method shown in FIG. 6 starts at step 610 by generating an initial training dataset that includes all the data acquired from the plurality of detectors. In step 620, the autoencoder is trained using the generated training dataset. Then, in step 630, the trained autoencoder is applied to all the data acquired from the plurality of detectors to derive outputs. In step 640, it is determined whether the outputs from the autoencoder are stable. Here, “stable” can mean there is no significant change in the outliers identified by the autoencoder based on latent vectors or reconstructed maps, or there is no significant change in distribution of the latent vectors or reconstruction error of the autoencoder.

If the answer at step 640 is “No,” the initial training dataset can be filtered to keep only high confidence normal data. In other words, only high confidence normal data will be used as a training dataset in a next iteration of training. Preferably, clustering is used in the selection of the training datasets. Further, a narrow cut can be used to keep only data well falling within a main cluster of the latent vectors or reconstruction error, for example, within ±0.5 standard deviations of the main cluster.

Steps 620-650 are repeated until stable outputs are derived from the autoencoder. At this point, the training of the autoencoder and the identification of anomalies are finalized. In a non-limiting example, the process shown in FIG. 6 is conducted with a set of input data (for example, a Lu background spectrum) obtained from a PET system to automatically train the autoencoder and identify defects.

In accordance with the methods described in this disclosure, either reconstructed data from the autoencoder or latent vectors calculated by the autoencoder are used to detect defects.

In one embodiment, to identify defects, the data reconstructed by the autoencoder is used to identify defects automatically, manually, or through another AE. In a non-limiting example, a pre-set threshold can be applied to automatically identify the image error. Firstly, the sum of squares difference (SSE) between the reconstructed data and the input can be calculated. Then, by comparing the calculated SSE with the threshold, non-uniform (i.e., defective) systems can be differentiated from uniform systems. As another non-limiting example, the data reconstructed from the autoencoder can be presented to an expert user (e.g., an operator or a service person who is responsible for reviewing quality-control images) to perform manual identification of defects. Alternatively, another autoencoder can be used to classify images as “normal” or “abnormal”. This autoencoder can be pre-trained using training datasets consisting of AE-reconstructed data.

In another embodiment, the latent vectors calculated by the autoencoder are directly used. Because the calculated latent vectors are highly compressed, computational analysis and comparisons can be faster and easier. For example, when specifications for the expected values of all or some of the latent vectors are set, a direct analysis of the compressed vector data can be conducted. As another example, clustering can be applied to the latent vectors, such as a k-means clustering algorithm. Alternatively, a power spectrum analysis or a principal-components analysis can be used.

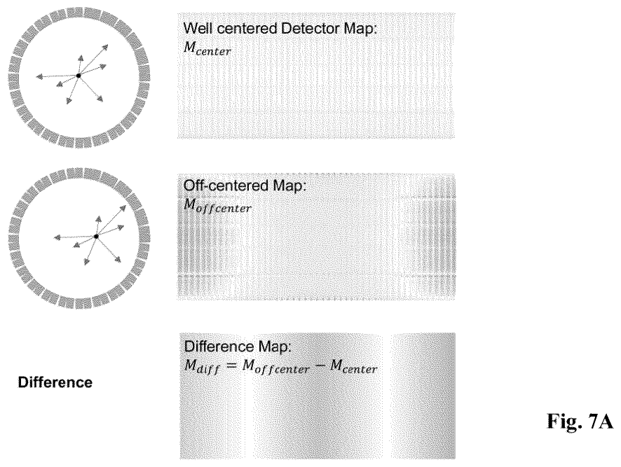

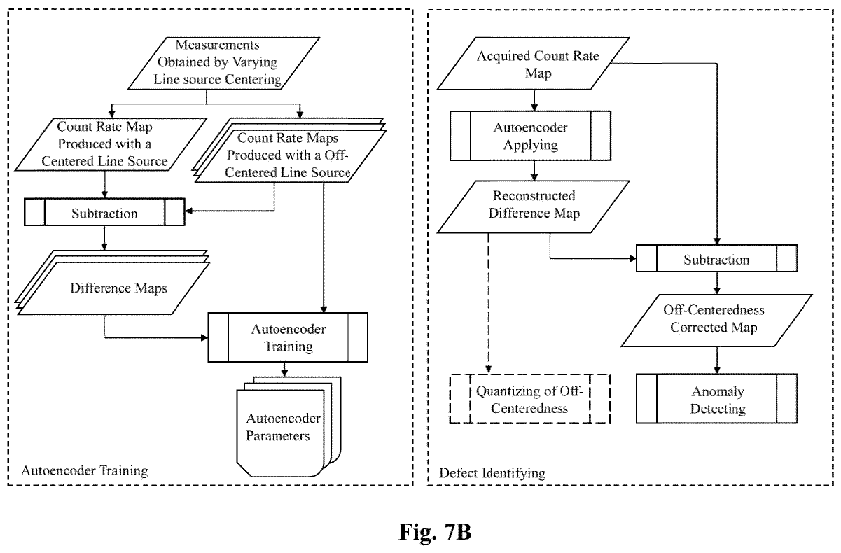

FIG. 7A-7B show a non-limiting example of identifying a specific change and/or correcting its impacts, according to one embodiment of the present disclosure. In this example, an autoencoder learns to identify and reconstruct specific changes in the system. The trained autoencoder can be used in change monitoring, trouble shooting, or data correction.

As one example of system changes, FIG. 7A shows different positions of a line source with respect to a detector ring. As shown in FIG. 7A, when the line source is off entered, the measured count rate map will have a non-uniform distribution, which could impair the capability to identify real problems of the system.

An autoencoder can be trained to overcome such an impairment. For each singles count rate map measured with an off-centered line source, a difference map compared to the singles count rate map measured with a well-centered line source can be produced. Using the difference maps as an input and the off-centered maps as a target, the autoencoder can learn to reconstruct the difference map.

Thus, when a measured count rate map is inputted to the trained autoencoder, the autoencoder can reconstruct a difference map. Thus, a corrected count rate map can be generated based on the reconstructed difference map and the measured count rate map.

On the basis of the corrected count rate map, defect detection can be carried out to identify the “real” anomalies in the system. The identification can be done by the same autoencoder, or by another autoencoder. Additionally or alternatively, the extent of the off-centeredness may be quantified from analysis of the reconstructed difference map.

Although the embodiments of this disclosure are described in the context of a whole system with a multitude of independent detectors, autoencoder-based defect detection can be applied to an individual detector, as part of a manufacturing process. By collecting plenty of data from a multitude of similar detectors to train an autoencoder, and operating the autoencoder on a single detector, performance of the detector can be evaluated.

Compared with the known approaches, autoencoder-based defect detection in accordance with the embodiments of the present disclosure uses machine learning to summarize the detector characteristics, without having any knowledge regarding the meaning of the acquired data, such as detector maps. An autoencoder takes over from a human or empirical algorithm the responsibility of determining what characteristics are most defining among normal and abnormal behaviors. Due to its automatic nature, the defect detection according to the embodiments of the present disclosure is more robustly automated. The autoencoder is able to detect unusual phenomena for which an empirical algorithm might not have been designed.

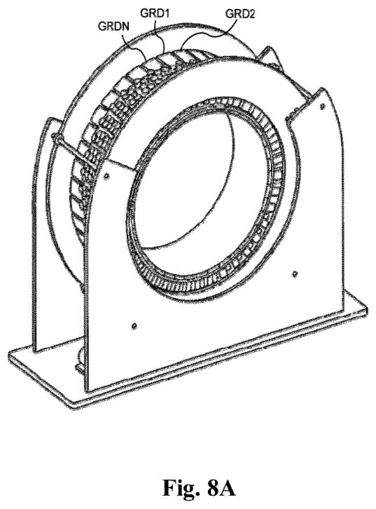

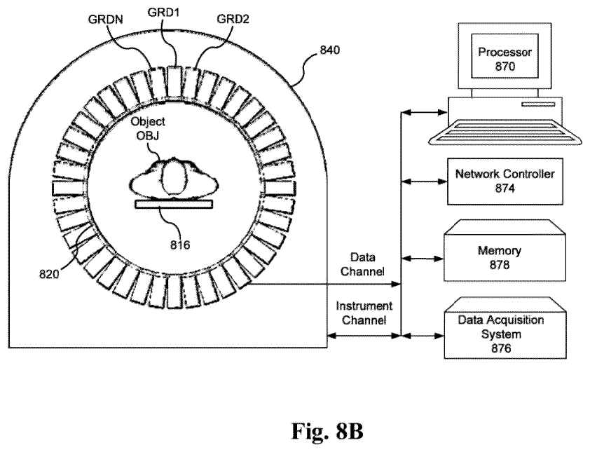

FIGS. 8A and 8B illustrate in implementation in which a medical imaging system includes a PET scanner that can implement the methods described in this disclosure. The PET scanner includes a plurality of gamma-ray detectors (GRDs) (e.g., GRD1, GRD2, through GRDN) that are each configured as rectangular detector modules.

Each GRD can include a two-dimensional array of individual detector crystals, which absorb gamma radiation and emit scintillation photons. The scintillation photons can be detected by a two-dimensional array of photomultiplier tubes (PMTs) or silicon photomultipliers (SiPMs). A light guide can be disposed between the array of detector crystals and the photodetectors.

Each photodetector (e.g., PMT or SiPM) can produce an analog signal that indicates when scintillation events occur, and an energy of the gamma ray producing the detection event. Moreover, the photons emitted from one detector crystal can be detected by more than one photodetector, and, based on the analog signal produced at each photodetector, the detector crystal corresponding to the detection event can be determined using Anger logic and crystal decoding, for example.

FIG. 8B shows one example of the arrangement of the PET scanner, in which the object OBJ to be imaged rests on a table 816 and the GRD modules GRD1 through GRDN are arranged circumferentially around the object OBJ and the table 816. The GRDs can be fixedly connected to a circular component 820 that is fixedly connected to a gantry 840. The gantry 840 houses many parts of the PET scanner. The gantry 840 of the PET scanner also includes an open aperture through which the object OBJ and the table 816 can pass, and gamma-rays emitted in opposite directions from the object OBJ due to an annihilation event can be detected by the GRDs and timing and energy information can be used to determine coincidences for gamma-ray pairs.

In FIG. 8B, circuitry and hardware are also shown for acquiring, storing, processing, and distributing gamma-ray detection data. The circuitry and hardware include: a processor 870, a network controller 874, a memory 878, and a data acquisition system (DAS) 876. The PET scanner also includes a data channel that routes detection measurement results from the GRDs to the DAS 876, the processor 870, the memory 878, and the network controller 874. The data acquisition system 876 can control the acquisition, digitization, and routing of the detection data from the detectors. In one implementation, the DAS 876 controls the movement of the bed 816. The processor 870 performs functions including reconstructing images from the detection data, pre-reconstruction processing of the detection data, and post-reconstruction processing of the image data, as discussed herein.

The processor 870 can be configured to perform various steps of the methods described herein and variations thereof. The processor 870 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 870 can execute a computer program including a set of computer-readable instructions that perform various steps of the described methods, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 878 can be a hard disk drive, CD-ROM drive, DVD drive, FLASH drive, RAM, ROM or any other electronic storage known in the art.

The network controller 874, such as an Intel Ethernet PRO network interface card from Intel Corporation of America, can interface between the various parts of the PET scanner. Additionally, the network controller 874 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

Embodiments of the present disclosure may also be as set forth in the following parentheticals.
(1) A method for detecting an anomaly related to a medical imaging device, the method comprising: acquiring data from a plurality of detectors of the medical imaging device; applying the acquired data to a first autoencoder; and detecting, based on outputs from the first autoencoder, an anomaly related to the medical imaging device.
(2) The method according to (1), wherein the first autoencoder comprises an encoder configured to generate latent vectors, and a decoder configured to reconstruct data from the generated latent vectors, and the step of detecting the anomaly further comprises detecting, based on the generated latent vectors or the reconstructed data, the anomaly related to the medical imaging device.
(3) The method according to any one of (1) and (2), further comprising: training the first autoencoder based on a training dataset generated from the data acquired from the plurality of detectors.
(4) The method according to any one of (1)-(3), wherein the step of applying the acquired data further comprises: splitting, based on a predetermined criterion, the acquired data into a first group of data and a second group of data, applying the first group of data to the first autoencoder to obtain first outputs, and applying the second group of data to the first autoencoder to obtain second outputs; and the step of detecting the anomaly further comprises detecting, based on a difference between the first outputs and the second outputs, the anomaly related to the medical imaging device.
(5) The method according to (4), wherein the predetermined criterion is an operating condition of the medical imaging device, the first and second groups of data correspond to a first operating condition and a second operating condition, respectively, and the first and second operating conditions are different from each other.
(6) The method according to (4), wherein the predetermined criterion is a processing method of the data acquired from the plurality of detectors, the first and second groups of data correspond to a first processing method and a second processing method, respectively, and the first and second processing methods are different from each other.
(7) The method according to (4), wherein the predetermined criterion is a time at which the data from the plurality of detectors is acquired, the first and second groups of data correspond to a first time and a second time, respectively, and the first and second times are different from each other.
(8) The method according to (4), wherein the step of training the first autoencoder further comprises: splitting, based on the predetermined criterion, the generated training dataset into a first training dataset and a second training dataset; and training the first autoencoder using the first training dataset and the second training dataset.
(9) The method according to 3, wherein the step of training the first autoencoder further comprises: generating the training dataset, which comprise all the data acquired from the plurality of detectors; training the first autoencoder based on the generated training dataset; operating, on all the data acquired from the plurality of detectors, the trained first autoencoder to derive outputs therefrom; determining whether the derived outputs are stable; and when the derived outputs are not stable, filtering the training dataset to generate an updated training dataset, and using the updated training dataset to repeat the training, operating, and determining steps until stable outputs are derived from the first autoencoder, so as to finalize the training thereof.
(10) The method according to (9), wherein the determining step further comprises: detecting, based on the derived outputs, an anomaly related to the medical imaging device; and determining that the derived outputs are stable when a difference between the anomalies detected in a last two iterations is less than a threshold.
(11) The method according to (9), wherein the determining step further comprises: performing clustering on the derived outputs to obtain a distribution of the derived outputs; and determining that the derived outputs are stable when a difference between the distributions obtained in a last two iterations is less than a threshold.
(12) The method according to (11), wherein the filtering is based on a standard deviation of a main cluster produced in the step of performing clustering.
(13) The method according to any one of (1)-(12), wherein the detecting step further comprises detecting the anomaly: by means of direct analysis of the outputs from the first autoencoder, by means of clustering of the outputs from the first autoencoder, or by means of a power spectrum analysis of the outputs from the first autoencoder.
(14) The method according to any one of (1)-(13), further comprising: applying the acquired data to a second autoencoder, identifying, based on outputs from the second autoencoder, a defect in a calibration process of the medical imaging device and correcting, based on the outputs from the second autoencoder, the acquired data to offset an effect of the identified defect on the medical imaging device, and wherein the step of applying the acquired data to the first autoencoder further comprises applying the corrected data to the first autoencoder, and the step of detecting the anomaly further comprises detecting, based on outputs from the first autoencoder with respect to the corrected data, the anomaly related to the medical imaging device.
(15) The method according to (14), further comprising: quantizing, based on the outputs from the second autoencoder, a magnitude of the identified defect.
(16) The method according to any one of (14) and (15), further comprising: training the second autoencoder based on a training dataset generated from the data acquired from the plurality of detectors.
(17) The method according to any one of (14)-(16), wherein the identified defect is off-centeredness of a radiation source used in the calibration process.
(18) A method for detecting an anomaly in a detector of a medical imaging device, comprising: acquiring data from the detector, applying the acquired data to an autoencoder, and detecting, based on outputs from the autoencoder, an anomaly in the detector.
(19) The method according to (18), further comprising training the autoencoder based on a training dataset which is generated from data collected from a plurality of detectors of a same type as the detector.
(20) An apparatus for detecting an anomaly related to a medical imaging device, the apparatus comprising processing circuitry configured to acquire data from a plurality of detectors of the medical imaging device, apply the acquired data to an autoencoder, and detect, based on outputs from the autoencoder, an anomaly related to the medical imaging device.

Those skilled in the art will also understand that there can be many variations made to the operations of the techniques explained above while still achieving the same objectives of the invention. Such variations are intended to be covered by the scope of this disclosure. As such, the foregoing descriptions of embodiments of the invention are not intended to be limiting. Rather. any limitations to embodiments of the invention are presented in the following claims.

What is claimed is:

1. A method for detecting an anomaly related to a medical imaging device, the method comprising:
acquiring data from a plurality of detectors of the medical imaging device;
applying the acquired data to a first autoencoder; and
detecting, based on outputs from the first autoencoder, an anomaly related to the medical imaging device.

2. The method of claim 1, wherein the first autoencoder comprises an encoder configured to generate latent vectors, and a decoder configured to reconstruct data from the generated latent vectors, and the step of detecting the anomaly further comprises detecting, based on the generated latent vectors or the reconstructed data, the anomaly related to the medical imaging device.

3. The method of claim 1, further comprising:
training the first autoencoder based on a training dataset generated from the data acquired from the plurality of detectors.

4. The method of claim 3, wherein the step of applying the acquired data further comprises:
splitting, based on a predetermined criterion, the acquired data into a first group of data and a second group of data,
applying the first group of data to the first autoencoder to obtain first outputs, and
applying the second group of data to the first autoencoder to obtain second outputs; and
the step of detecting the anomaly further comprises detecting, based on a difference between the first outputs and the second outputs, the anomaly related to the medical imaging device.

5. The method of claim 4, wherein
the predetermined criterion is an operating condition of the medical imaging device,
the first and second groups of data correspond to a first operating condition and a second operating condition, respectively, and
the first and second operating conditions are different from each other.

6. The method of claim 4, wherein
the predetermined criterion is a processing method of the data acquired from the plurality of detectors,
the first and second groups of data correspond to a first processing method and a second processing method, respectively, and
the first and second processing methods are different from each other.

7. The method of claim 4, wherein
the predetermined criterion is a time at which the data from the plurality of detectors is acquired,
the first and second groups of data correspond to a first time and a second time, respectively, and
the first and second times are different from each other.

8. The method of claim 4, wherein the step of training the first autoencoder further comprises:
splitting, based on the predetermined criterion, the generated training dataset into a first training dataset and a second training dataset; and
training the first autoencoder using the first training dataset and the second training dataset.

9. The method of claim 3, wherein the step of training the first autoencoder further comprises:
generating the training dataset, which comprise all the data acquired from the plurality of detectors;
training the first autoencoder based on the generated training dataset;
operating, on all the data acquired from the plurality of detectors, the trained first autoencoder to derive outputs therefrom;
determining whether the derived outputs are stable; and
when the derived outputs are not stable,
filtering the training dataset to generate an updated training dataset, and
using the updated training dataset to repeat the training, operating, and determining steps until stable outputs are derived from the first autoencoder, so as to finalize the training thereof.

10. The method of claim 9, wherein the determining step further comprises:
detecting, based on the derived outputs, an anomaly related to the medical imaging device; and
determining that the derived outputs are stable when a difference between the anomalies detected in a last two iterations is less than a threshold.

11. The method of claim 9, wherein the determining step further comprises:
performing clustering on the derived outputs to obtain a distribution of the derived outputs; and
determining that the derived outputs are stable when a difference between the distributions obtained in a last two iterations is less than a threshold.

12. The method of claim 11, wherein the filtering is based on a standard deviation of a main cluster produced in the step of performing clustering.

13. The method of claim 1, wherein the detecting step further comprises detecting the anomaly:
by means of direct analysis of the outputs from the first autoencoder;
by means of clustering of the outputs from the first autoencoder; or
by means of a power spectrum analysis of the outputs from the first autoencoder.

14. The method of claim 1, further comprising:
applying the acquired data to a second autoencoder;
identifying, based on outputs from the second autoencoder, a defect in a calibration process of the medical imaging device; and
correcting, based on the outputs from the second autoencoder, the acquired data to offset an effect of the identified defect on the medical imaging device, and wherein
the step of applying the acquired data to the first autoencoder further comprises applying the corrected data to the first autoencoder, and
the step of detecting the anomaly further comprises detecting, based on outputs from the first autoencoder with respect to the corrected data, the anomaly related to the medical imaging device.

15. The method of claim 14, further comprising:
quantizing, based on the outputs from the second autoencoder, a magnitude of the identified defect.

16. The method of claim 14, further comprising:
training the second autoencoder based on a training dataset generated from the data acquired from the plurality of detectors.

17. The method of claim 14, wherein the identified defect is off-centeredness of a radiation source used in the calibration process.

18. A method for detecting an anomaly in a detector of a medical imaging device, comprising:
acquiring data from the detector;
applying the acquired data to an autoencoder; and
detecting, based on outputs from the autoencoder, an anomaly in the detector.

19. The method of claim 18, further comprising:
training the autoencoder based on a training dataset which is generated from data collected from a plurality of detectors of a same type as the detector.

20. An apparatus for detecting an anomaly related to a medical imaging device, the apparatus comprising
processing circuitry configured to
acquire data from a plurality of detectors of the medical imaging device;
apply the acquired data to an autoencoder, and
detect, based on outputs from the autoencoder, an anomaly related to the medical imaging device.
